(19) United States
(12) Patent Application Publication
(10) Pub. No.: US 2016/0103122 A1
(43) Pub. Date: Apr. 14, 2016

(54) METHOD FOR THE CREATION OF A DATABASE FOR INITIAL ASSESSMENT OF THE EFFECTIVENESS OF ACTIVE AGENTS IN TUMOR THERAPY

(71) Applicant: FLACOD GMBH, Heidelberg (DE)

(72) Inventor: Christof GRANZOW, Heidelberg (DE)

(73) Assignee: Flacod GmbH, Heidelberg (DE)

(21) Appl. No.: 14/895,056

(22) PCT Filed: Jun. 10, 2014
(86) PCT No.: PCT/DE2014/000293

§ 371 Date: Dec. 1, 2015

(30) Foreign Application Priority Data
Jun. 25, 2013 (DE) 10 2013 010 528.5

(51) Int. Cl. G01N 33/50; G06F 19/00

(52) U.S. Cl. CPC G01N 33/5011; G06F 19/3443; G06F 19/3487; G01N 2500/10; G01N 2800/52; G01N 2800/7028

(57) ABSTRACT

The method comprises the steps wherein the tumour cells from a tumor tissue sample previously taken from a particular patient are incubated ex vivo with the active agent concerned and the IC50 value thereof for these cells is determined; wherein the same patient is treated with this active agent in accordance with evidence-based therapy rules; wherein the therapy outcome is documented and classified; and wherein the documentation and/or classification of the therapy outcome obtained is assigned to the IC50 value determined ex vivo.

The invention relates to a method for creating a database for the initial assessment (pre-assessment, making a prognosis) of the efficacy of a (cytostatic) active substance or a combination of (cytostatic) active substances in the chemotherapy of a tumor of a particular patient, and to procedures for ex vivo pre-testing (initial testing) of a scheduled active substance or combination of active substances and predicting its efficacy (therapeutic effect) on the tumor of a particular patient by using this database.

Malignancies (synonym: cancerous tumors) are malignant tumors which are deprived of the normal growth control of the organism, proliferate unrestricted, invade the surrounding tissue and destroy it, and which can metastasize, i.e. they can penetrate into blood and lymphatic vessels and via these may reach other body organs where they colonize again and continue to proliferate (i.e. form metastases).

In contrast, benign tumors are characterized by the fact that they do not grow invasively into the surrounding tissue, but merely displace it, and that they do not form metastases.

Although in the malignancy therapy (synonym: cancer therapy), treatment with radiation or with intake of cytostatic agents or a combination of both methods is used for decades, the success rate is still only about 20 percent for most cancers.

Particularly in the case of chemotherapy there is the problem that treatment with an inappropriate (false) or under-dosed chemotherapeutic agent does not only have no inhibiting effect on the malignant tumor, but also can lead to increased resistance of the malignant tumor to treatment.

As a consequence, in the case of two or more cycles of chemotherapy with the same chemotherapeutic agent, which is a common measure in the art, the success rates after the second and further treatment are usually much lower than after the first treatment.

Currently, the vast majority of treatment schedules in chemotherapy and radiotherapy are defined more or less empirically. This is problematic because each malignant tumor of an individual reacts differently to a chemotherapeutic agent and/or to radiation.

For a therapy which is effective and tailored to the specific malignant tumor of an individual test methods have been already proposed with which prior to administration of a scheduled chemotherapy and/or radiotherapy the sensitivity of malignant cells to the scheduled therapeutic agent, particularly the chemotherapeutic agent, can be examined, for example in US 20010051353 A1 and WO 2009124997 A1.

The implementation of such a test procedure is performed in vitro, preferably ex vivo, that is on a malignant tumor tissue sample freshly isolated from the organism such as described in WO 2009124997 A1 or DE 102012003700 B3 (while US 20010051353 A1 proposes and uses cell monolayer cultures that have been cultivated and expanded over several weeks.)

By incubation with various concentrations of the therapeutic agent, its IC50-value can be determined. The IC50-value of a chemotherapeutic agent is the concentration of said agent by which the proliferation of the treated and studied cells is reduced to 50% in comparison to untreated control cells. The IC50-value is an important criterion for describing the response of a malignant tumor to a chemotherapeutic agent.

On the basis of the test results it is possible to estimate the degree of sensibility of the malignant cells for the therapeutic agent.

It has been demonstrated in recent years that not only the malignant cells of a malignant tumor are capable of developing resistance, but also, or even only, the cells of the malignant tumor's stroma, e.g in primary and metastatic lung tumors (Granzow et al. 2004) and in head and neck carcinomas (Doliner et al. 2004). Hence, prior to incubation with the active substance to be tested the tumor tissue samples should be subjected to a procedure for identifying—and possibly also separating—and differentiating malignant cells on the one hand and stromal cells (especially endothelial cells and fibroblasts) on the other hand. Thus, the respective reaction of the respective group of cells to the active substance tested may be examined subsequently.

The results of such testing of the efficacy of the active substance, in particular the IC50-values of the malignant tumor cells of the tumor tissue sample of the patient in question, are used in the prior art as a rough indication of the type of treatment schedule regarding dosage and administration cycles that should be chosen for said patient to achieve the desired therapeutic effect.

Because chemotherapy indeed, harbors the chances to inhibit the tumor significantly, but also is accompanied by a substantial toxicity for other cells of the thus treated organism (i.e. the patient), it should be adapted to the patient and his tumor concerned as accurate as possible.

Both, the nature and the dosage of the applied active substance or the applied active substances/substance combination should be selected in a way that on the one hand the tumor is brought to regression or at least growth arrest with high probability, and on the other hand the rest of the cells of the patient's organism are burdened with toxicity as little as possible. In practice, however, appropriate data or information sources for such a patient-specific, individualized treatment design that enable a quick and targeted access to each individual case of a specific patient, are lacking so far.

It is an object of the present invention to eliminate these disadvantages of the prior art or at least to mitigate them.

One achievement of this object is the provision of a method for creating a database for the initial assessment (pre-assessment) of the efficacy of a (cytostatic) active substance in the therapeutic treatment of a tumor—in particular a malignant tumor and more particularly a carcinoma—of a particular patient, based on the ex-vivo determination of the IC50-value of the active substance for the proper tumor cells (malignant cells) of a previously obtained tumor tissue sample from the same patient. This method is characterized by the following measures:
Cells of the tumor which are capable of proliferation ex vivo, i.e. both the malignant tumor cells of the tumor tissue sample previously obtained as well as the stromal cells from this tumor tissue sample, are incubated together (either in the same batch or in parallel batches) ex vivo with the active substance in question.
The IC50-value of the active substance is determined at least in the malignant cells.
The same patient is treated with the active substance in accordance with current evidence-based treatment rules.
The treatment results are documented and classified, for example in the form of a classification with the classes I=complete remission of the tumor (CR), II=partial remission of the tumor (PR), III=zero growth of the tumor=“no change” (NC), and IV=progressive tumor growth=Progress.
The documentation and/or classification of the resultant therapeutic result/result/outcome is assigned to the IC50-value as pre-determined ex vivo.

Definitions in the present context:

The term “malignant cells” refers to cells of a malignant tumor, which proliferate autonomously, are capable of invasive and destructive growth, and may form metastases.

The term “stromal cells” refers to the cells of a malignant tumor which, in vivo, co-exist with the malignant cells, but do not themselves possess biological malignancy, and which are able to proliferate ex vivo (e.g. fibroblasts and cellular components of the blood vessel system, in particular endothelial cells).

The term “cell cluster” (plural: “cell clusters”) refers to largely homogeneous cellular aggregations of cells, which similarly exist or have existed in the tissue of a malignant tumor in situ.

The term “tumor digest” refers to the collagenase digestion product obtained from the tumor tissue sample in vivo by centrifugation and resuspension in culture medium.

The term “cell line” refers to a primary culture (i.e. the culturing of cells derived from cells, tissues or organs) at the time of the first subculture.

The term “cell strain” refers to cells that have been obtained by selection or cloning from a primary culture or from a cell line, and that exhibit characteristics which persist during a subsequent cultivation or subcultivation.

The abbreviation “g” represents the average gravitational acceleration.

The term “IC50 value” represents the mean inhibitory concentration (IC50) of an inhibitor (generally speaking: an active substance), i.e. the concentration at which half-maximal inhibition is observed. According to FDA the IC50 is the concentration of a substance that is necessary to achieve a 50% inhibition at a target in vitro. In case of colony formation by cells from a tumor tissue sample (cell colony) the IC50-value is the concentration of an active substance/inhibitor at which a half-maximal inhibition of colony formation (of the respective cell type) is observed as compared to negative controls (0 μM active substance).

The concentration data “M”, “mM” and “μM” refer to moles per liter, millimoles per liter and micromoles per liter.

Compared to the prior art, the database of the present invention allows a significantly improved method for initial assessing (pre-assessing) the efficacy of a (cytostatic) active substance to combat a tumor of a particular patient, based on the ex vivo determination of the IC50-value of this active substance for the tumor cells of the patient concerned. This is especially true when the tumor is a malignant tumor and more particularly a carcinoma. In this improved method, the tumor cells of a previously removed tumor tissue sample from a current patient are incubated ex vivo with the active substance in question, and the IC50-value of this active substance is determined for the malignant cells.

Then the determined IC50-value is compared to the reference IC50-values contained in the database and having been determined previously for the same active substance on tumor cells of the same tumor entity.

As stated above, in the database each reference IC50-value is associated with a therapy outcome that has been obtained (or is obtainable) by that the patient for whose tumor cells the reference IC50-value has been (or will be) obtained, was (or will be) treated with the active substance according to applicable evidence-based therapy rules, and that the resulting therapy outcome was (or will be) documented and classified, for example in the form of a classification in one of the classes I=complete remission of the tumor (CR), II=partial remission of tumor (PR), III=zero growth of the tumor=“no change” (NC), and IV=progressive tumor growth=Progress.

A match between the IC50-value determined for the tumor cells of the current patient and any of the reference IC50-value contained in the database is considered to be an indication that the therapy outcome assigned in the database to this reference value will be achieved in the case of treatment of the current patient, too.

Hence, a further solution of the above mentioned object is the provision of such a method for initial assessing (pre-assessing) of the efficacy of a (cytostatic) active substance to combat a tumor.

A solution of the aforementioned object is also the use of the database according to the invention in a process for initial assessing (pre-assessing) of the efficacy of a (cytostatic) agent which is scheduled or intended to combat a tumor of a particular patient or comes/could come into consideration for this purpose.

Instead of the IC50-value in the given teachings to create the database and their use, it is, in principle, possible to use another parameter for the efficacy of the active substance, provided this parameter describes the relationship between molar concentration and toxicity to living cells ex vivo in a measurable and reproducible manner.

The preparation of a tumor tissue specimen for the incubation with the active substance to be tested preferably comprises the following measures:

The preferably freshly harvested tumor tissue sample is first (preferably within 15 minutes maximum) cleaned of necrotic portions and attaching other tissue (not of malignant tumor) and minced (preferably cut), preferably in pieces of about 1 to 4 mm³ (cubic millimeter) volume.

The minced tissue is suspended in (preferably serum-containing) culture medium. The suspension obtained is subjected to treatment with collagenase (i.e. a collagenase digestion, or collagenase decomposition) at about 36° C. to 37° C. The collagenase digestion product thus obtained is centrifuged (e.g. 5 minutes at 50×g and room temperature), and the recovered pellet is separated from the supernatant.

This pellet is resuspended in culture medium and the tumor digest thus obtained is subsequently incubated together with the active substance. Here the chronological order is of importance by which the aliquots of the tumor digests are added to the previously prepared equally large but double concentrated aliquots of active substance in culture medium.

In a preferred embodiment according to this method for determining the IC50 the usually mathematically (on the basis of manufacturer's instructions for the stock solution) constructed concentrations of the active-substance-in-culture-medium aliquots are rechecked in a verification test.

This verification test is characterized in that from some dilution levels of dilution series prepared from the stock solution of the active ingredient and culture medium, aliquots are removed for the incubation and IC50-determination using the cells of the tumor digests, as well as for a parallel IC50-determination using cells of an established cell strain or an established cell line each from a primary culture of cells of the same tumor entity as the tumor digest. All test approaches, that is, those using the cells of the tumor digests and those using the cells of the established cell line or cell strain, are in parallel (simultaneously) incubated, and analyzed with regard to their IC50-value by using the same analytical methods.

For a separate treatment or testing of malignant cells on the one hand and/or stromal cells (especially endothelial cells and fibroblasts) on the other hand and, in addition, each in the form of cell clusters, the pellet prior to incubation with the active substance to be tested may be subjected to a separation process according to DE 102012003700B3.

The invention will be explained in more detail on the basis of the following embodiments and accompanying tables:
Table 1 shows: For 18 lung cancer (bronchial carcinoma) samples (column 1 “serial nos. 1-18.”) from 17 different patients, the pathohistological classification and the tumor stage (column 3), the cytostatic agents used in the chemotherapy administered to the patient (column 4) and the patients' clinically diagnosed therapy outcome (column 5).
   SCLC=small cell lung cancer
   NSCLC=non-small cell lung cancer
   CarboPt=carboplatin
   CisPt=cisplatin
   PR=partial response (i.e. the carcinoma has become detectably smaller)
   NC=“No Change”=zero growth of the carcinoma
   Progress=the carcinoma has continued to grow, no therapeutic success
   Tumors ID 278 and ID 279 came from the same patient. They were independently existing, non-small cell primary tumors. ID 278 was an adenocarcinoma, ID 279 a squamous cell carcinoma.
Table 2 shows: The correlation of the pretherapeutically ex vivo determined IC50-values for cisplatin of the tumor cells to the outcome of the platinum-based chemotherapy of the patients in vivo. Distinctive constellations are:
   (Patient) group (1):
   low IC50 values (below 5 μM cisplatin), i.e.: high sensitivity (sensibility) of the cells to this active substance;
   Partial remission (PR) of the tumor, i.e.: good therapeutic success;
   (Patient) group (2):
   intermediate IC50-values (greater than or equal to 5 and smaller than 8 μM cisplatin) i.e.: moderate sensitivity (sensibility) of the cells to this active substance;
   Partial remission (PR) or zero growth (NC=“no change”) of the tumor, i.e.: therapeutic success;
   (Patient) group (3):
      high IC50-values (8 to 25.6 μM cisplatin), i.e.: low sensitivity of the cells to that active substance; nevertheless partial remission (PR) or zero growth (NC) of the tumor, i.e.: therapeutic success;
   (Patient) group (4):
   contains special cases
Table 3 shows: The graphical representation of the database “Treatment outcome prognosis in lung cancer (bronchial carcinoma) with the cytostatic drug cisplatin”

### EXAMPLE 1

### Creation of a Database for Initial Assessment (Pre-Assessment) of the Efficacy of the Cytostatic Agent Cisplatin in the Chemotherapeutic Treatment of Bronchial Carcinoma

(I) Determination of the IC50 for Cisplatin in Tumor Cells of Lung Cancer (Bronchial Carcinoma) Tissue Samples Obtained from Patients with Untreated Bronchial Carcinoma.

Parts of freshly (under general anesthesia of the patient) taken (here biopsied) lung cancer (bronchial carcinoma) tissue were transported to the laboratory and processed immediately for IC50 determination.

The transport to the laboratory should take place in chilled culture medium. For this purpose in particular HEPES- and NaHCO₃-buffered, preferably flavin-free RPMI 1640 culture medium containing 10% fetal bovine serum, 100 IU/mL penicillin G, 100 ug/mL streptomycin, 20 μg/mL Amikacin (as antibiotics) and 8 μl/mL Voriconazole (as an antifungal agent) are suitable.

In the laboratory, this tumor tissue samples were cleaned, weighed, minced and suspended in culture medium.

For this purpose in particular the method according to DE 102012003700 B3 is suitable, wherein about 5.5 mg tumor tissue sample per 1 mL of culture medium are subjected to enzymatic digestion with collagenase, preferably by using Clostridium histolytikum collagenase (Sigma, 230 CDU/mL), in a Petri dish under standard conditions in a gassing incubator (36-37° C., 3.5% CO₂ in water vapor saturated air) for 4-5 hours.

Following the collagenase digestion, the digestion product is centrifuged at 50×g, the supernatant is discarded and the pellet is resuspended in 1 mL of fresh culture medium. With this resuspension—hereinafter referred to as “tumor digest”—the chemosensitivity test (IC50 determination) is performed

For the chemosensitivity assay (IC50-determination) with cisplatin, initially cisplatin solutions were prepared as follows: from a 1 mM stock solution of cisplatin in water test solutions in culture medium were prepared with the following final concentrations of cisplatin: 0 μM (as a negative control); 0.1 μM; 0.2 μM; 0.4 μM; 0.8 μM; 1, 6 μM; 3.2 μM; 6.4 μM; 12.8 μM and 25.6 μM. These cisplatin concentrations calculated on the basis of manufacturer's specifications on the stock solution were verified in a verification test by IC50-determination.

For IC50 determination in the tumor digests (cell suspensions from tumor tissue samples of the patient) in a 96-well culture plate coated with extracellular matrix (ECM) a duplicate (two wells) per tumor digest was prepared for each final concentration of cisplatin, where in each of the two cavities 150 μl cisplatin test solution of the same final concentration of cisplatin were submitted.

The thus prepared culture plates were pre-incubated for about 1 hour under standard conditions (i.e.: gassing incubator; 36-37° C.; 3.5% CO₂ in water vapor saturated air). Subsequently, 150 μl tumor digest were added to these pre-incubated wells, they were incubated for 72 hours under standard conditions (see above), and then analyzed microscopically.

For the verification test of the applied cisplatin concentrations, a cell suspension of lung adenocarcinoma cell strain A240286S (Heuser et al., 2005) with a cell density of 3×10⁴/mL was used instead of the patients' tumor digests. From each of the initial solutions with 0 μM, 0.4 μM, 0.8 μM, 1.6 μM, 3.2 μM and 6.4 μM cisplatin used for the tumor digests, in duplicates per concentration indicated 150 μl each were filled per cavity of a 96-well culture plate suitable for cell culturing. The final concentration gradients were pre-incubated for approximately 30 minutes under standard conditions (see above). Subsequently, per cavity each 150 μl of a A240286S single cell suspension (cell density: 3×10⁴/mL) freshly prepared with accutase (preferably Promocell) was added to the cisplatin solutions previously filled in. The culture plate was then incubated, in parallel with the culture plate fitted with the tumor digests, for 72 hours under standard conditions.

All work with serum-containing culture medium and cisplatin were performed at a workplace illumination exclusively with light of wavelengths above 520 nm.

After the end of incubation, in all tumor digest test preparations the culture medium was removed, and the existing cell colonies were rinsed with phosphate buffered saline (PBS) and drained. Subsequently, the cells were fixed with methanol at minus (−) 25° C. for 5 to 10 minutes and then subjected to Giemsa staining according to Gurr. The average number of existing colonies of epithelial tumor cells per cavity was determined microscopically for each cisplatin concentration including 0 μM cisplatin (as a negative control).

In the batches of the verification test (with the cells of the lung adenocarcinoma cell strain A240286S) the culture medium was decanted after the end of incubation. The present cellular layers were rinsed with PBS, drained and then removed from the culture vessel with Accutase. The cells were isolated by passing them through a blunt cannula no. 20. Using a cell-counting-and-analyzing apparatus (here the Casy I Cell Analyzer from Roche, Basel), the cell density was determined for each test batch and reduced by the inoculum.

For the determination of the cisplatin IC50-value the logarithm of both, the microscopically determined values (for the cells from the tumor digest) as well as the values determined by means of cell-counting and—of analyzer apparatus (for the cells of the verification tests), was taken and graphically shown (plotted) in comparison to the percentage of the negative control values (0 μM cisplatin). Subsequently, with log-linear procedures (in this example with methods of log-linear regression using the software SAS JMP Version 9.0) the IC50-values were determined in those segments of the plots in which the relative abundance of colonies linearly decreased with increasing concentration of cisplatin.

The thus obtained cisplatin IC50-values for the tumor cells (from tumor tissue samples) of the respective patients are shown in Table 2, column 2 in each of the patients' groups 1 to 4.

The IC50-values of the groups 1, 2 and 3 were opposed to each other as groups and compared using unpaired, two-sided t-tests. The resulting p-values are given in Table 2. Group 1 and Group 2 as well as Group 2 and Group 3 show significant differences to a two-sided significance level of 0.025 adjusted for multiple testing (this corresponds to Bonferroni-adjusted levels of 0.05/2). This means that the differences between Group 1 and Group 2, as well as between Group 2 and Group 3 are significant, the latter implying that Group 1 also is significantly different from Group 3. The probability of error is below one percent in each case.

The obtained IC50-values for cisplatin of A240286S cells in the verification tests amounted to average 1.2 μM±0.28 μM in 15 evaluable test preparations.

### (II) Pathohistological Classification, Tumor Stage, Chemotherapy and Outcome of the Therapeutic Treatment

In parallel to but independent of the determination of the IC50-values for cisplatin of the tumor cells of their tumor tissue samples in the laboratory, the respective patients were subjected to a platinum-based chemotherapy according to evidence-based protocols. According to these protocols cisplatin or carboplatin is—and consequently was in this example—always combined with one or more non-platinum cytostatic drugs, in this example with one or more of the active substances docetaxel, etoposide, gemcitabine, pemetrexed, LY2603618 or vinorelbine.

Cisplatin and carboplatin belong to the group of cytostatic platinum derivatives/platinum analogues that disturb cell proliferation by inducing breaks or cross links of the DNA strands. Vinorelbine and docetaxel inhibit the cell cycle, by engaging in formation or degradation of the mitotic spindle during mitosis.

Etoposide disrupts DNA replication by inhibiting topoisomerase II.

Gemcitabine after activation inhibits DNA synthesis as a nucleoside antimetabolite.

Pemetrexed inhibits several folate-dependent key enzymes of the de novo biosynthesis of thymidine and purine nucleotides.

The study substance LY2603618 is a ChK 1 inhibitor which disturbs the DNA repair mechanism.

The therapy outcome, namely the change or the state of the tumor after two to three cycles of treatment compared to the state before the start of treatment, has been analyzes by computer tomography and classified on the basis of the “standard”—rating scale known and conventional in the art as class I=complete remission (CR), class II=partial remission (PR), class III=zero growth (“no change”, NC) and class IV=Progress (progressive tumor growth).

The therapy outcomes achieved are shown in table 1, column 5 and in table 2 in column 3 of each group (1 to 4). Column 4 of table 1 shows with which cytostatic active substances the patient concerned had been treated.

In table 2 for each patient the cisplatin IC50-values determined with the cells of his tumor tissue sample ex vivo (before chemotherapy starts) are contrasted to the in vivo and in situ chemotherapy (with cisplatin or carboplatin combinations) outcome of his tumor. This comparison shows:
Ex-vivo determined IC50-values below 5 μM (patients' Group 1) correlate with pronounced platinum sensitivity of the tumor in vivo: all tumors in situ show partial remission (PR) after or during chemotherapy.
Ex-vivo determined IC50-values greater than or equal to 5 and below or equal to 8 μM (Group 2 of the patients studied) correlate with moderate platinum sensitivity of the tumor in vivo: The respective tumors in situ show either a partial response (PR) or at least zero growth (NC, “no change”) after or during chemotherapy.

The results ex-vivo and in-vivo for tumor tissue samples ID 278 and ID 279, both of which originate from the same patient (but of two different tumors for this patient), particularly illustrate that Group 1 is distinguishable from Group 2 (of the patients studied), i. e. that pronounced platinum sensitivity (Group 1) is distinguishable from moderate platinum sensitivity (Group 2): The IC50-value of the tumor cells of the tumor tissue sample ID 279 (of a squamous cell carcinoma) was 3.73 μM, and the respective tumor in vivo (the squamous cell carcinoma) showed after chemotherapy (with cisplatin combined with gemcitabine) a long lasting partial remission. By contrast, the IC50-value of the tumor cells of the tumor tissue sample ID 278 (of an adenocarcinoma) was 5.25 μM and the respective tumor in vivo (the coexisting adenocarcinoma) showed after the same chemotherapy (with cisplatin combined with gemcitabine) only zero growth.

The IC50-values of the seven tumors in Group 2 (moderate platinum sensitivity) are close to the average of 6.73 μM. In the prior art known pharmacokinetic studies have shown that the mean maximum blood plasma concentration of cisplatin tolerated by the patient in a five-day infusion treatment is 6.67 μM (Desoize et al., 1996).

Taking into account the natural variations in the blood plasma concentration of the substance, already for tumors with ex vivo determined IC50 values for cisplatin above 6.7 μM an incipient insensitivity (resistance) of the tumor in vivo and in situ (in the patient) to cisplatin is to be expected.

The results listed in Group 3 reveal patients' tumors whose cells ex vivo/in vitro featured a IC50-value for cisplatin above 8 μM.

Yet here the tumors in question as well as ID 228 from patients' Group 4 in vivo and in situ showed after chemotherapy with cisplatin (in combination with gemcitabine or etoposide or pemetrexed and partly other non-platinum cytostatic drugs) a therapeutic outcome in the form of partial remission or a zero growth of the tumor.

An explanation for this observation is that in these six cases the therapeutic effects/successes (PR, NC) are based on the cooperation of the non-platinum cytostatic substances pemtrexed (ID 228, ID 297, ID 311, ID 314), etoposide (ID 272) and gemcitabine (ID 258) with cisplatin or carboplatin.

Pretherapeutic IC50-determinations for cisplatin or other platinum analogues should be complemented by assays that demonstrate whether platinum resistant bronchial (lung) carcinoma cell contained in the tumor sample can be made platinum sensitive ex vivo with the help of non-platinum cytostatic agents, especially pemetrexed, etoposide and gemcitabine.

Group (4) comprises special cases:

For ID 233 and ID 228, the unambiguous mathematical determination of IC50-values by the log-linear plots was not possible; they were above 26 μM.

In the case of ID 282, chemotherapy was carried out solely with gemcitabine and without the use of platinum derivatives/platinum analogues

### (III) Database Creation and Information Content of the Database

The data or outcomes determined in (I) and (II) provide the following information: Given that ex vivo in laboratory tests (preferably with the procedure according to I) the IC50-values for cisplatin of the epithelial tumor cells of a bronchial carcinoma (lung cancer) tissue sample of any current patient are below 5 μM, it is expected that a cisplatin treatment or a treatment with other platinum derivatives/platinum analogues results in partial remission of the tumor (bronchial carcinoma/lung cancer) in the patient concerned.

Given these IC50-values are between 5 μM and 6.7 μM, this suggests that chemotherapy of the patient concerned with cisplatin or other platinum derivatives/platinum analogues leads to at least arrest of growth of the tumor (bronchial carcinoma/lung cancer) with high probability. IC50-values for cisplatin above 6.7 μM mean resistance of the tumor in vivo and in situ against sole treatment with cisplatin.

According to the invention with this information it is possible to create a database for assessing the success of a planned chemotherapy based on platinum derivatives/platinum analogues, especially cisplatin, for the purpose of combating a bronchial carcinoma (lung cancer) (“therapy outcome prognosis in lung cancer with the cytostatic drug cisplatin”). At present this database provides the information

1) that an IC50-value for cisplatin below 5 μM (IC50<5 μM), which was determined in the laboratory, i.e. ex vivo with tumor cells (from a previously removed bronchial carcinoma/lung cancer tissue sample) of the patient concerned, correlates with the chemotherapy outcome prognosis “partial remission” or “tumor stage Class II”, if cisplatin is administered to this patient; and
2.) that an IC50-value for cisplatin between 5 μM and 6.7 μM (5 μM<IC50<6.7 μM), which was determined in the laboratory, i.e. ex vivo and in particular in vivo, with tumor cells (from a previously removed bronchial carcinoma/lung cancer tissue sample) of the patient concerned, correlates with chemotherapy outcome prognosis at least “zero growth” or at least “tumor stage class III” if cisplatin is administered to this patient;
3.) that an IC50-value for cisplatin above 6.7 μM indicates resistance of the corresponding tumor in the patient against a treatment solely with cisplatin. Therapeutic successes such as partial remission or zero growth of the tumor are then accessible only by combining cisplatin with other cytostatic drugs such as gemcitabine, etoposide and pemetrexed.

Further data collection according to the present description of the invention can confirm and/or specify and/or modify this hypothesis.

The same applies to the additional inclusion of sensitizers such as gemcitabine, etoposide and pemetrexed in the IC50-determination for cisplatin or other platinum analogues.

By feeding more data into this database, the correlation between IC50-value and chemotherapy outcome prognosis may be specified further, for example in subclasses or between classes of standard classes I, II, III and IV.

A possible representation of the database according to the invention “Therapy outcome prognosis with lung cancer (bronchial carcinoma) and administration of the cytostatic drug cisplatin” with the currently available data and information on the correlation between IC50-values (ex vivo/in vitro) and chemotherapy outcome (in vivo/in situ) is given in the form of table 3.

Platinum compounds form the basis for chemotherapy not only in lung cancer (bronchial carcinoma) but also in many other epithelial cancers (tumors). Thereby the amount of platinum dose tolerated by the patient depends upon the toxicity of the substance for central organism (body) functions, such as the formation of white blood cells in bone marrow. The individually existing tumor entity is irrelevant.

As a consequence, it is expected that the quantitative relations between the cisplatin sensitivity of the tumor cells ex vivo (measurable in laboratory testing) and the sensitivity of the tumor in vivo towards cisplatin-based chemotherapy of the patient determined in practice for the bronchial carcinoma (lung cancer), apply as well for other tumors treatable with cisplatin compounds in humans (and possibly animal).

A database according to the invention created by (I) and (II) thus provides a means to the responsible therapist to obtain, in relatively simple manner and in a sufficiently short time, a meaningful indication whether a scheduled platinum-based, particularly cisplatin-based therapy in the relevant individual bronchial carcinoma (lung cancer) of the individual patient concerned can be expected to be successful, and to what extent (tendency “merely” a halt in tumor growth, or tendency “remission/regression” of the tumor).

Analogously created databases for other tumor entities represent appropriate tools for each different tumor entity.

Conceivably, it may turn out that at least such tumor entities, which previously have shown great similarities with bronchial carcinoma (lung cancer), e.g. the ovarian cancer, reveal practically the same correlations between the ex vivo/in vitro determined IC50-values and the tumor stage in vivo and in situ after administered chemotherapy, especially in the case of platinum-based chemotherapy.

Literature Cited:
WO 2009/124997 A1 “Method and kit for the ex vivo evaluation of the response of a tumor to conditions to be tested”
DE 102012003700 B3 “Verfahren zur Trennung von Verbanden maligner Zellen und Verbänden aus Stromazellen einer Malignomgewebeprobe”
Dollner R, Granzow C, Helmke B M, Ruess A, Schad A, Dietz A: The impact of stromal cell contamination on chemosensitivity testing of head and neck carcinoma. Anticancer Res. 24: 325-31 (2004)
Granzow C, Kopun M, Heuser M, Herth F, Becker H D: Chemoresistance of human lung tumor stromal cells. Amer. Assn. Cancer Res. 95^{th} Annual Meeting Proc. Suppl., abstract LB-82 (2004)
Heuser M, Kopun M, Rittgen W, Granzow C: Cytotoxicity determination without photochemical artefacts. Cancer Lett. 223: 57-66 (2005)
Desoize B, Berthiot G, Manot L, Coninx P, Dumont P: Evaluation of a prediction model of cisplatin dose based on total platinum plasma concentration. Eur J Cancer 32A:1734-8 (1996)

TABLE 1
Pathohistological classification, tumor stage, chemotherapy
administered, and therapy outcome of 18 bronchial
carcinoma (lung cancer) tumor samples of 17 patients

| Serial | | Pathohistology/ | Cytostatic drug | Therapy |
|---|---|---|---|---|
| No. | ID | Tumor Stage “St.” | administered | outcome |
| 1 | 220 | SCLC/St. IV | CarboPt, Etoposid | PR |
| 2 | 221 | NSCLC/St. IV | CisPt, Gemcitabine | PR |
| 3 | 228 | NSCLC/St. IV | CarboPt, Pemetrexed | PR |
| 4 | 233 | NSCLC/St. IV | CisPt, CarboPt, Etoposid, | Progress |
| | | | Docetaxel | |
| 5 | 245 | NSCLC/St. IV | CarboPt, Gemcitabine | PR |
| 6 | 255 | SCLC/St. IV | CarboPt, Etoposid | PR |
| 7 | 258 | NSCLC/St. IV | CisPt, Gemcitabine | PR |
| 8 | 260 | NSCLC/St. IIIb | CisPt, CarboPt, | PR |
| | | | Gemcitabine | |
| 9 | 272 | SCLC/St. IV | CisPt, Etoposid | NC |
| 10 | 275 | NSCLC/St. IV | CarboPt, Gemcitabine | NC |
| 11 | 277 | NSCLC/St. IIIa | CarboPt, Vinorelbin | NC |
| 12 | 278 | NSCLC/St. IV | CisPt, Gemcitabine | NC |
| 13 | 279 | NSCLC/St. IV | CisPt, Gemcitabine | PR |
| 14 | 282 | NSCLC/St. IV | Gemcitabine | Progress |
| 15 | 294 | NSCLC/St. IV | CarboPt, Pemetrexed | NC |
| 16 | 297 | NSCLC/St. IV | CisPt, Pemetrexed, | NC |
| | | | LY2603618 | |
| 17 | 311 | NSCLC/KI. IIIb | CisPt, Pemetrexed | NC |
| 18 | 314 | NSCLC/KI. IV | CisPt, CarboPt, | PR |
| | | | Pemetrexed | |

TABLE 2
Correlation of the pretherapeutically ex vivo determined IC50-values for
cisplatin of tumor cells to the outcomes of the platinum-based
chemotherapy in patients in vivo
Cisplatin response ex vivo

| Group 1 | | | Group 2 | | | Group 3 | | | Group 4 | | |
|---|---|---|---|---|---|---|---|---|---|---|---|
| IC50 < 5 μM | | | IC50 ≧ 5 < 8 μM | | | IC50 ≧ 8 bis 25.6 μM | | | Others | | |
| | | Chemo- | | | Chemo- | | | Chemo- | | | Chemo- |
| ID | IC50 | response | ID | IC50 | response | ID | IC50 | response | ID | IC50 | response |
| 220 | 2.60 | PR | 278 | 5.25 | NC | 258 | 16.72 | PR | 282 | 14.8 | Progress |
| 279 | 3.73 | PR | 275 | 5.38 | NC | 314 | 20.51 | PR | 228 | >26 | PR |
| 255 | 4.58 | PR | 260 | 5.61 | PR | 272 | 21.01 | NC | 233 | >26 | Progress |
| | | | 277 | 7.58 | NC | 311 | 22.09 | NC | | | |
| | | | 294 | 7.70 | NC | 297 | 25.60 | NC | | | |
| | | | 245 | 7.74 | PR | | | | | | |
| | | | 221 | 7.77 | PR | | | | | | |

| | | |
|---|---|---|
| mean value 3.64 | mean value 6.72 | mean value 21.12 |
| Standard deviation | Standard deviation | Standard deviation |
| 0.99 | 1.23 | 3.19 |
| p-value group 1 vs. 2 = | p-value group 2 vs. 3 = | |
| 0.0095 | 0.0002 | |

TABLE 3
Database “Therapy outcome prognosis for bronchial carcinoma
(lung cancer) and administration of the cytostatic drug cisplatin”

| Pretherapeutically ex vivo/in vitro | Prognosis of the in vivo-effect of the |
|---|---|
| determined IC50-values for | Cisplatin-based chemotherapy in |
| Cisplatin of malignant tumor cells | patients |
| 0 μM < IC50 < . . . ? . . . μM | class I |
| | (not observed hitherto) |
| . . . ? . . . μM < IC50 < 5 μM | class II |
| 5 μM ≦ IC50 ≦ 6.7 μM | class II-III |
| | (combination therapy is needed) |
| 6.7 μM < IC50 < . . . ? . . . | class II-IV |
| | (combination therapy is needed) |

Class I = complete remission of the tumor (CR),
Class II = partial remission of the tumor (PR)
Class III = zero growth of the tumor = “no change” (NC)
Class IV = progressive tumor growth = Progress

1. A method for creating a database for the initial assessment (pre-assessment) of the efficacy of a cytostatic active substance (cytostatic drug) in the chemotherapeutic treatment of a tumor, especially a malignant tumor, of a particular patient on the basis of ex vivo determination of the IC50 value of the active substance for the tumor cells of a previously obtained tumor tissue sample from the same patient, comprising the following measures:
the tumor cells (especially the malignant cells) of the previously obtained tumor tissue sample (or malignant tissue sample) of the patient are incubated ex vivo with the active substance concerned,
the IC50 value of this active substance in these cells is determined,
the ex vivo determined IC50-value is assigned to the documentation and/or classification of the therapy outcome obtained with the same patient after treatment according to applicable evidence based therapy rules with the same active substance.

2. The method according to claim 1, wherein the incubation of the tumor cells of the tumor tissue sample of the patient with the active substance ex vivo comprises the following steps in the given order:
preparation of a tumor digest from the tumor tissue sample by means of collagenase digestion, centrifuging and resuspending in culture medium,
addition of aliquots of this tumor digests to previously prepared twice concentrated equal aliquots of active substance in culture medium;
verification of the concentrations of the active substances used by means of investigation, in parallel, of established in vitro cell strains of the same tumor entity.

3. The method according to claim 1, wherein the cells used to determine the IC50 value are malignant cells previously separated ex vivo from the removed tumor tissue sample.

4. (canceled)

5. A method for the initial assessment (pre-assessment) of the efficacy of an active (cytostatic) substance (drug) in the therapeutic treatment of a tumor, particularly a malignant tumor, of a specific (certain) current patient, wherein
the tumor cells of a previously obtained tumor tissue sample from the patient are incubated ex vivo with the active substance concerned and the IC50 value of this active substance is determined in these cells,
the determined IC50 value is compared with previously determined reference IC50 values of the same active substance for tumor cells of the same tumor entity, wherein each reference IC50 value is assigned a therapy outcome obtained from that patient whose tumor cells had been used for obtaining said reference IC50 value after a treatment with the active substance according to applicable evidence-based therapy rules,
and a match between the IC50 value of the tumor cells of the current patient and a reference IC50 value is considered to be an indication that the therapy outcome assigned to this reference value will also occur in the case of treatment of the current patient.

6. The method according to claim 1, wherein the classification of the therapy outcome is given in the form of classes I to IV, wherein classes I=complete remission of the tumor (CR), II=partial remission of the tumor (PR), III=zero growth of the tumor=“no change” (NC), and IV=progressive tumor growth=Progress.

7. The method according to claim 1, wherein the active cytostatic substance is a platinum derivative/platinum analogoue/platinum compound.

8. The method according to claim 1, wherein the tumor is lung cancer (bronchial carcinoma).

9. The method according to claim 5, wherein the incubation of the tumor cells of the tumor tissue sample of the patient with the active substance ex vivo comprises the following steps in the given order:
preparation of a tumor digest from the tumor tissue sample by means of collagenase digestion, centrifugation and resuspension in culture medium,
addition of aliquots of this tumor digest to previously prepared twice concentrated equal aliquots of active substance in culture medium;
verification of the concentrations of the active substance used by means of investigation, in parallel, of established in vitro cell strains of the same tumor entity.

10. The method according to claim 5, characterized in that the cells used to determine the IC50-value are malignant cells previously separated ex vivo from the obtained tumor tissue sample.

11. The method according to claim 5, wherein the resulting therapy outcome has been documented and classified in one of the classes I=complete remission of the tumor (CR), II=partial remission of the tumor (PR), III=zero growth of the tumor=“no change” (NC), and IV=progressive tumor growth=Progress.

12. The method according to claim 5, wherein the active cytostatic substance is a platinum derivative/platinum analogoue/platinum compound.

13. The method according to claim 5, wherein the tumor is lung cancer (bronchial carcinoma).

14. A method of selecting a platinum derivative/platinum analogoue/platinum compound as active substance or part of a combination of active substances in a chemotherapy predictive of partial response or zero growth of a tumor, particularly a malignant tumor, in a specific/certain patient, comprising:
incubating the tumor cells (especially the malignant cells) of a previously obtained tumor tissue sample of the patient ex vivo with the platinum derivative/platinum analogoue/platinum compound and determining the IC50-value of this active substance in these patient's cells,
comparing the obtained ex vivo determined IC50-value with previously determined reference IC50-values of the same active substance with tumor cells of the same tumor entity, wherein each of these reference IC50-values is assigned to a therapy outcome obtained from that patient whose tumor cells had been used for obtaining said reference IC50-value after chemotherapeutic treatment with the active substance according to applicable evidence-based therapy rules,
and wherein a match with a reference IC50-value is an indication that the therapy outcome assigned to this reference IC50-value will also occur in the case of treatment of said specific/certain patient's lung cancer (bronchial carcinoma) with a platinum derivative/platinum analogoue/platinum compound as active substance or part of a combination of active substances.

15. The method according to claim 14, wherein the incubation of the tumor cells of the tumor tissue sample of the patient with the active substance ex vivo comprises the following steps in the given order:
preparation of a tumor digest from the tumor tissue sample by means of collagenase digestion, centrifugation and resuspension in culture medium,
addition of aliquots of this tumor digests to previously prepared twice concentrated equal aliquots of active substance in culture medium;
verification of the concentrations of the active substance used by means of investigation, in parallel, of established in vitro cell strains of the same tumor entity.

16. The method according to claim 14, characterized in that the cells used to determine the IC50 value are malignant cells previously separated ex vivo from the obtained tumor tissue sample.

17. The method according to claim 14, wherein the tumor is lung cancer (bronchial carcinoma).

18. The method according to claim 14, wherein the resulting therapy outcome has been documented and classified in one of the classes I=complete remission of the tumor (CR), II=partial remission of the tumor (PR), III=zero growth of the tumor=“no change” (NC), and IV=progressive tumor growth=Progress.
